(19) United States
(12) Patent Application Publication
(10) Pub. No.: US 2013/0005958 A1
(43) Pub. Date: Jan. 3, 2013

(54) DEVICES AND METHODS FOR REDUCING RADIOLYSIS OF RADIOISOTOPES

(75) Inventors: Christian Friedrich Peter Rensch, Munich (DE); Marko Klaus Baller, Saarbruecken (DE); Christoph Boeld, Munich (DE); Ruben Julian Horvath-Klein, Munich (DE); Victor Donald Samper, Kirchseeon (DE); Johan Urban Ingemar Ulin, Uppsala (SE)

(73) Assignee: GENERAL ELECTRIC COMPANY, Schenectady, NY (US)

(21) Appl. No.: 13/173,912

(22) Filed: Jun. 30, 2011

(51) Int. Cl. C07H 5/02; B01D 15/08

(52) U.S. Cl. 536/122; 210/198.2

(57) ABSTRACT

Disclosed are devices and methods for reducing radiolysis of radiopharmaceuticals during filtration, concentration and purification. The devices comprises two or more confining geometry having a cross-section dimension below the beta(+) or beta(−) range of the radioisotope in use, when containing the radioisotope, and configured in such a way that neighboring geometries are isolated from its nearest neighbor such that no measurable kinetic positron energy transfer occurs between the confining geometries when containing the radioisotope. Methods of filtration of radioisotope containing mixtures are also disclosed.

### BACKGROUND

The invention relates generally to devices and methods for reducing radiolysis in the production and purification of radiopharmaceuticals.

Positron Emission Tomography (PET), together with Single Photon Emission Computed Tomography (SPECT), is a powerful medical imaging technology that is finding use in the expanding field of molecular imaging in medical diagnostics and drug discovery.

The application of microfluidics and related technologies for the synthesis of radiopharmaceuticals for Positron Emission Tomography (PET) has gained increasing attention in the scientific community. Benefits such as reduced reaction times, highly efficient reactions, low reagent consumption, reduced system footprint and increased system automation are of high interest and have been demonstrated. Further downscaling is anticipated, especially for radiolabeling reactions that may benefit from high concentrations of one reactant over the other as well as for research studies utilizing cost-intensive precursors.

The downscaling of synthesis reaction volumes for radiopharmaceutical production implies an increase of activity per unit volume. The conventional process of activity concentration into a given synthesis volume is ultimately limited by radiolysis. Radiolysis, and more specifically autoradiolysis, is the decomposition of molecules at high concentrations of radioactivity over time. As used herein, radiolysis, radiolytic effects and autoradiolysis may be used interchangeably.

Radiolytic effects arise from the ionization and dissociation cascade initiated by the isotope decay event and the positron (beta+) emission. They occur in the range of several millimeters, depending on the utilized isotope and the surrounding media. The direct disintegration and ionization of molecules along the ionization path of the emitted positron may lead to subsequent formation of free reactive species that interfere with the radiopharmaceutical compound of interest. This process reduces the amount of useful radiopharmaceutical molecules and increases the concentration of impurities in the product solution. Radiolysis occurs in all commonly utilized positron emitting PET radioisotopes such as ¹⁸F, ¹¹C and ⁶⁸Ga, however, autoradiolysis phenomena will vary depending on the respective positron energies for each type of isotopes.

Various national pharmacopoeias stipulate the minimum purity that a radiopharmaceutical product must meet at the time of injection to the patient. For example, ¹⁸F-fluoro-deoxy-glucose ([¹⁸F]FDG) typically has a minimum specification of greater than or equal to 95% purity; thereby defining the shelf life of the drug. Since such compounds sometimes have to be transferred from a production site to the customer, several techniques have been employed to increase the shelf life time.

To address radiolysis, certain techniques have been used to limit the interaction probability of free radicals with tracer molecules in a bulk solution. The techniques include dilution of the product, scavenging of free radicals by utilizing additives (e.g. ethanol) [Kiselev, M. Y., Tadino, V., inventors, 2006. Eastern Isotopes, Inc., Assignee. Stabilization of Radiopharmaceuticals Labeled with 18-F. U.S. Pat. No. 7,018,614] or freezing [Wahl et al. “Inhibition of Autoradiolysis of Radiolabeled Monoclonal Antibodies by Cryopreservation”; Journal of Nuclear Medicine Vol. 31 No. 1 84-89] of the solution thus reducing the diffusion of free radicals. However, these techniques represent an additional process step to be integrated into production hence increasing the overall level of synthesis complexity. Furthermore, conventional scavenging and stabilizing methods may not be applicable under all circumstances for existing and future radiopharmaceutical compounds, chemistry methods utilized during synthesis and purification as well as fluid volumes and activity concentrations. More specifically, with respect to purification, high local densities of radioactive species can occur, leading to an increased autoradiolysis rate in those regions.

Therefore an approach which reduces the radiolytic effects of radiopharmaceutical compounds without the use of additives through production, purification, and storage is desirable. Such an approach may include the reduction of autoradiolysis of radiopharmaceutical compounds by partial geometric reduction of the positron emission induced ionization and decomposition effects. Thus designing fluid confinement for the production, purification or storage of radiopharmaceutical compounds, wherein the geometric arrangement has a characteristic dimension below the beta+/beta− energy dissipation range of the utilized radioisotope may provide a means of increasing synthesis efficiency, radiochemical purity and the shelf life and efficacy of the radiopharmaceutical compounds.

### BRIEF DESCRIPTION

In one aspect, the present invention relates to devices and methods for filtering a radioisotope containing mixture. The devices comprise two or more confining geometries comprising an opening to allow fluid transfer in to said confining geometries, a cross-section dimension below the beta(+) or beta(−) range of a radioisotope, when containing the radioisotope; and adjacent confining geometry configured such that neighboring geometries are isolated from the nearest neighbor geometry such that no measurable kinetic positron energy transfer occurs between the geometries when containing the radioisotope.

In another aspect, the present invention relates to methods of filtering, concentrating and/or purifying radioisotope containing mixtures. The method comprising: adding the radioisotope containing mixture of to a filtering device, flowing the mixture through the device, wherein the flow rate is controlled to separate and purify the radioisotope compound from the mixture; and collecting sample from the outlet port of wherein the sample comprises the radioisotope. The filtering device comprising at least one confining geometry comprising an inlet port and an outlet port to allow fluid flow through said confining geometry; cross-section dimension of the fluid confining geometry is below the beta(+) or beta(−) range of a radioisotope, when containing the radioisotope; and wherein adjacent confining geometries are configured such that neighboring geometries are isolated from the nearest neighbor s such that no measurable kinetic positron energy transfer occurs between the geometries when containing the radioisotope;

### BRIEF DESCRIPTION OF THE FIGS

These and other features, aspects, and advantages of the present invention will become better understood when the following detailed description is read with reference to the accompanying figures wherein:

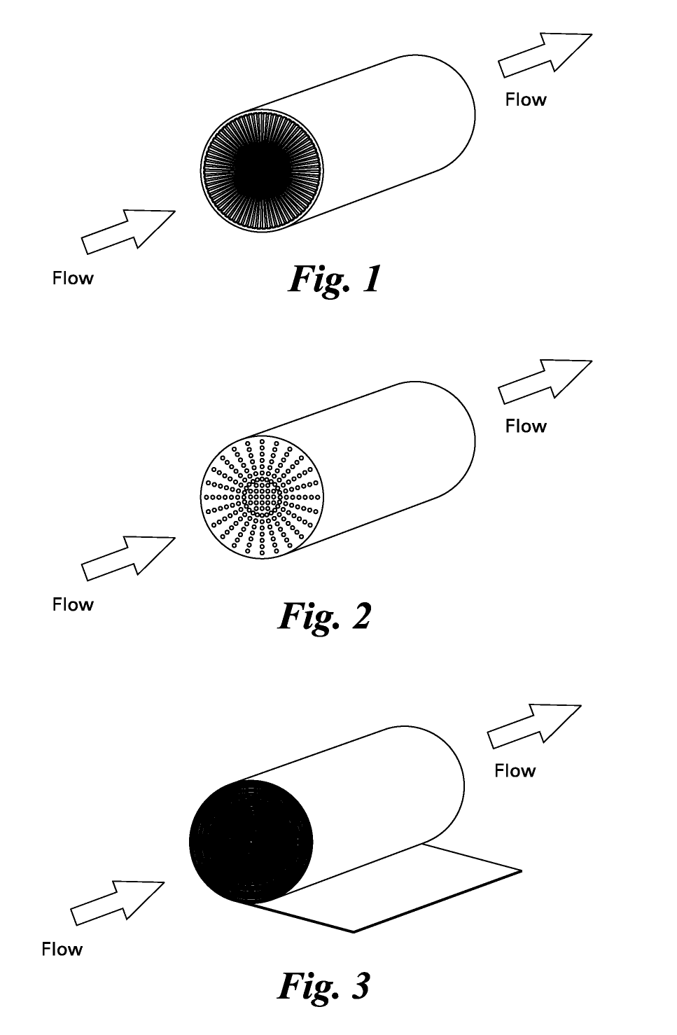

FIG. 1 is an illustration of a segmented column for filtration of radiopharmaceuticals.

FIG. 2 is an illustration of a segmented column for filtration of radiopharmaceuticals having capillary-sized through holes.

FIG. 3 is an illustration of a wrapped foil with surface coating/resin for filtration whereas the thickness of the foil and the coating is designed to compensate for positron interaction and subsequent autoradiolysis.

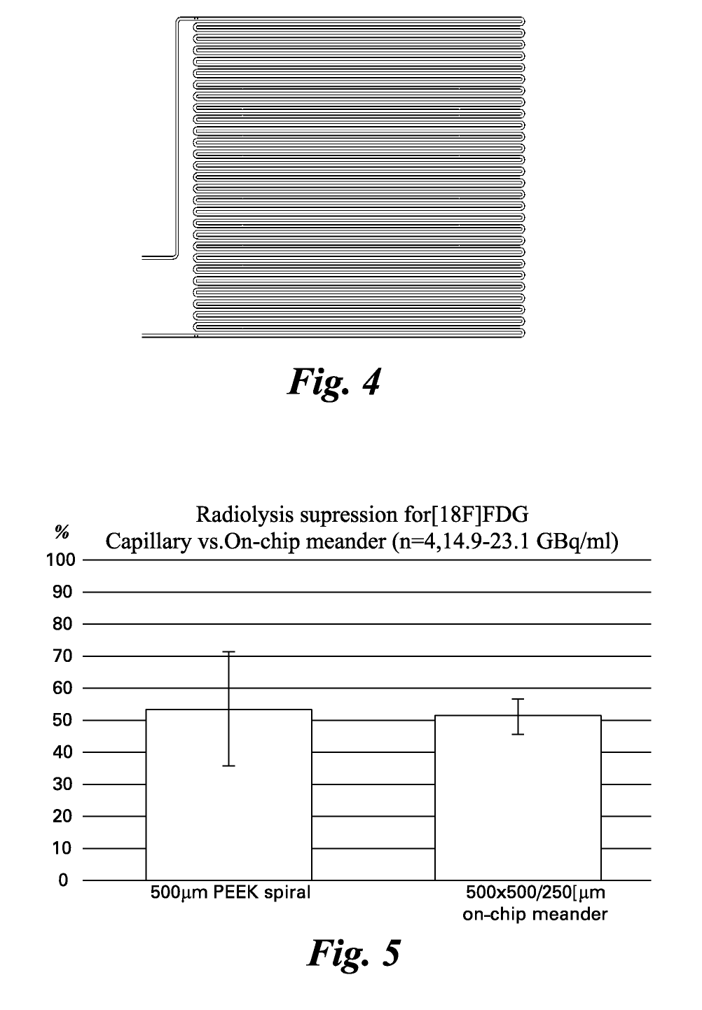

FIG. 4 an illustration of a top view of a microfluidic meander-shaped storage/reaction container with channel size 500 μm×500 μm, 250 μm spacing.

FIG. 5 shows experimental results for positron interaction between adjacent channels on a microfluidic chip with channel size 500 μm×500 μm, 250 μm spacing, utilizing [¹⁸F]FDG (non-stabilized) at 14.9-23.1 GBq/ml compared to a shielded PEEK capillary.

FIG. 6 is a graphical representation of the cumulative probability distribution T(x) for positron annihilation events in water.

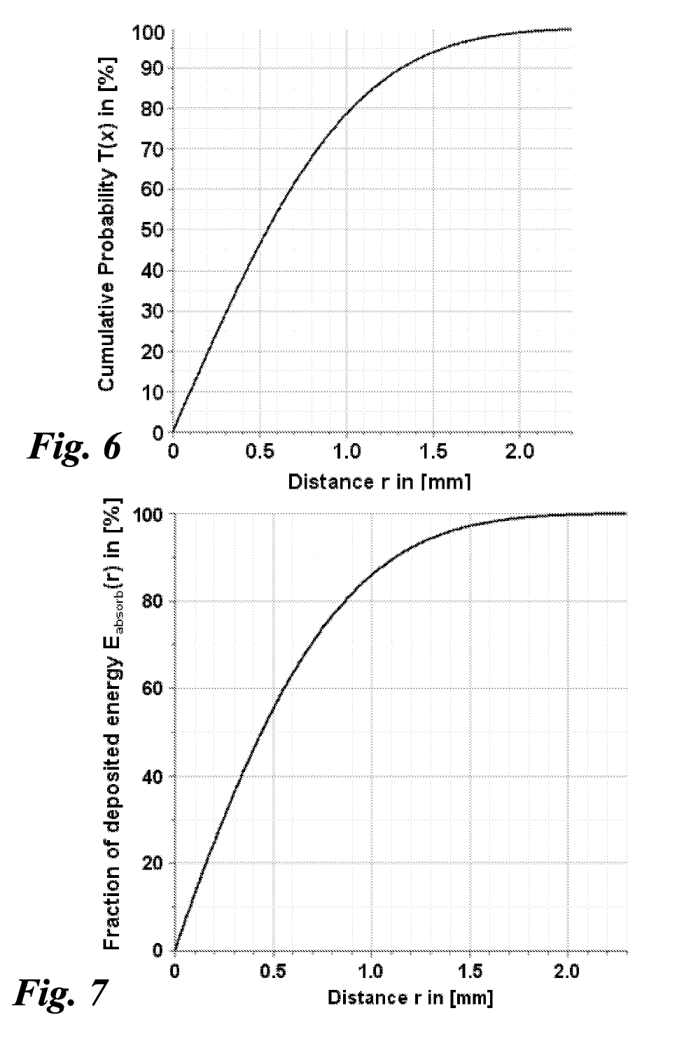

FIG. 7 is a graphical representation of fraction of deposited Energy E_{absorb}(r) for positrons in water.

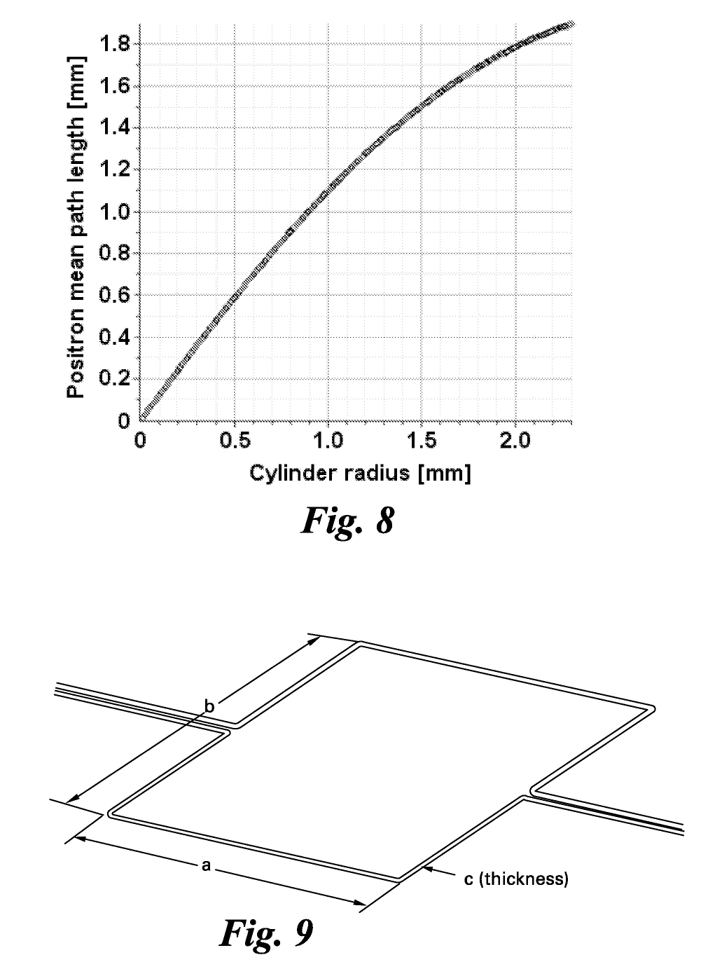

FIG. 8 is a graphical representation of mean path length as a function of radius for cylindrical geometries.

FIG. 9 is an illustration of a planar reactor with outer dimensions a, b, and thickness c.

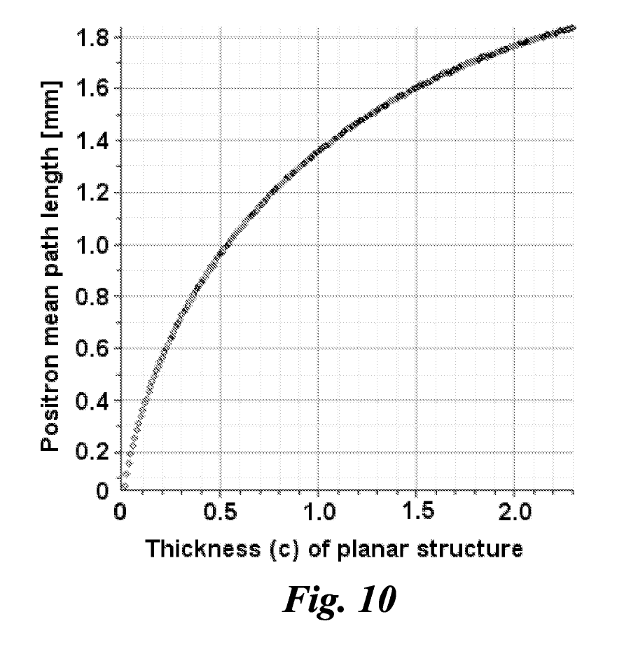

FIG. 10 is a graphical representation of mean path length in a planar geometry according to FIG. 9 as a function of the structure thickness c.

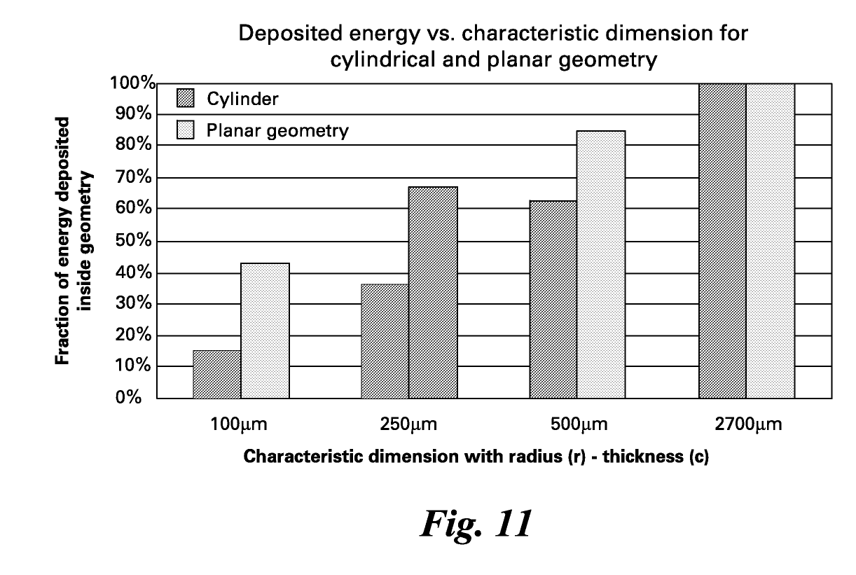

FIG. 11 is a graphical representation comparing fractional deposited energy inside a cylindrical versus a planar structure for varying characteristic dimensions (radius for a cylinder and thickness for a planar configuration).

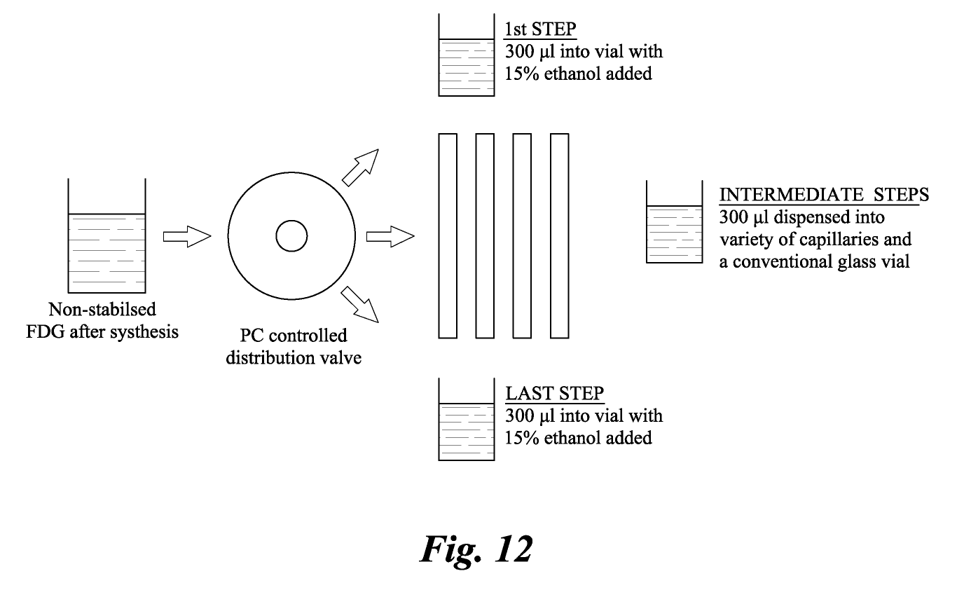

FIG. 12 is an illustration of the experimental set-up used.

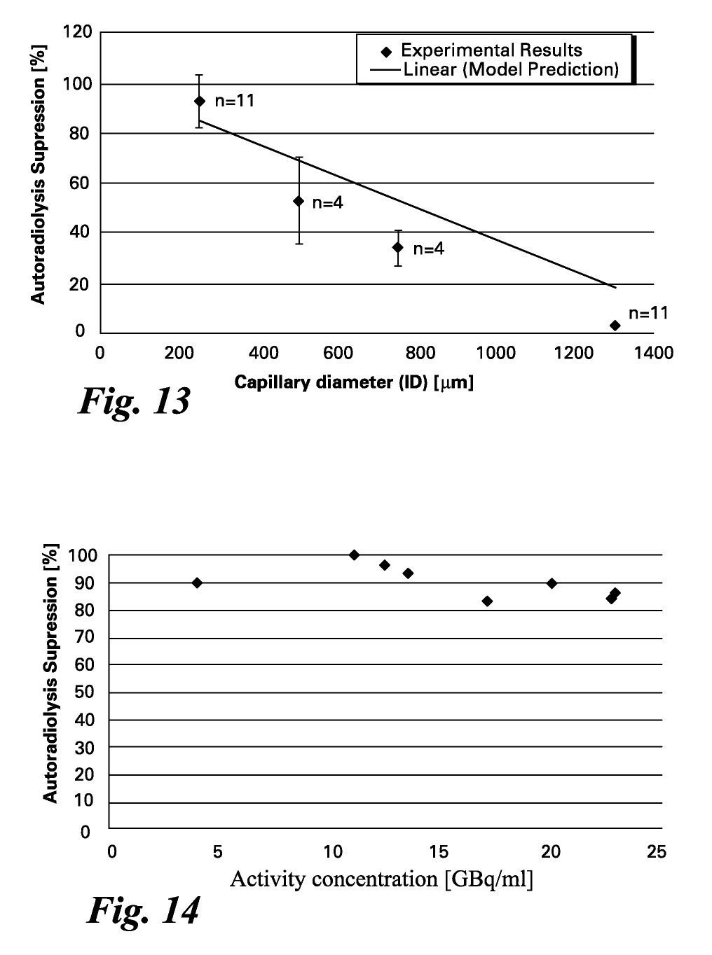

FIG. 13 graphically shows autoradiolysis suppression versus capillary diameter measured on several high activity (14.9-23.1 GBq/ml) experiments utilizing non-stabilized [18F]FDG.

FIG. 14 shows the autoradiolysis suppression in ID 250 μm PEEK capillary vs. activity concentration whereas yields show no significant correlation with the activity concentrations utilized during the experiment.

### DETAILED DESCRIPTION

The following detailed description is exemplary and not intended to limit the invention of the application and uses of the invention. Furthermore, there is no intention to be limited by any theory presented in the preceding background of the invention or descriptions of the drawings.

Positron Emission Tomography (PET), together with Single Photon Emission Computed Tomography (SPECT), is a powerful medical imaging technology that is building the foundation of a rapidly expanding field of molecular imaging in medical diagnostics and drug discovery. As such, there has been a growing body of research in the area of microfluidic synthesis of PET tracers. In addition to the promise of higher reaction yields and improved process control, microfluidics has the potential to reduce the infrastructure burden of PET by reducing the overall size and shielding of tracer synthesizers.

The scale-down of radiochemistry from typical reaction volumes in the area of approx. 1000 μl, to micro reactors of approximately 100 μl or smaller, leads to higher concentrations of activity if a single synthesis batch in order to produce the same amount of patient doses as the conventional equivalent process. However, it is known that with an increase of activity concentration, there is also a decrease in product yield and purity due to autoradiolysis. For example, in a conventional scale reactor with a diameter of ca. 10 mm and a volume of 10 ml, approximately 99% of the positrons' energy is dissipated in the liquid matter inside the reactor in a process that can lead to radiolysis.

Further with respect to microfluidics, autoradiolysis, which arises from the interaction of radical species created by positron interaction, may be reduced by surface modifications to getter radicals that lead to a permanent or temporary capturing/binding of radicals to a surface. Due to short diffusion lengths for particles in micro-channels, the probability of a radical reaching the wall a capillary tube or a microfluidic structure before interacting with a radiolabeled molecule of interest is higher than compared to a conventional vessel. Therefore, controlling variations in geometry and scale may alter the positron's degree of interaction with the reactor contents as well as the interaction of radical species induced by positron energy dissipation, and thus impact the radiolysis process. Thus the design of the fluid confining geometry for reactor vessels, purification, or storage devices may enable increase output activities and more effective production systems at increased product shelf life capabilities.

More specifically, in purifying and/or concentrating radioisotopes, high local densities of radioactive species may occur, leading to an increased autoradiolysis rate in those regions. Therefore design of purifying elements having specific confining geometries may alter the positron's degree of interaction with the confines of the purifying elements as well as the interaction of radical species induced by positron energy dissipation.

The invention relates generally to filtration devices for the purification and/or concentration of radioisotopes including, but not limited to radiopharmaceuticals. In certain embodiments, the devices comprises fluid or fluid guiding elements wherein the guiding elements, which may also be referred to as fluid confining geometries, have dimensions below the average beta+and beta− interaction range of emitting radioisotopes, which may be contained within the elements.

As used herein beta decay may be defined as a type of radioactive decay in which a beta particle, an electron or a positron, is emitted. Beta+ (β+) emission refers to positron emission; electron emission is referred to as beta−(β−)emission. The geometries of the filtration devices include a confining geometry such as channels or channel-like assemblies and refers to a capillary, trench or groove like structure through which a fluid may flow. The term confining geometry and channel is used interchangeably. In certain embodiments, the geometry of the elements may reduce autoradiolysis or radiolytic effects. Radiolytic effects or autoradiolysis include positron emission induced direct disruption of molecules as well as radical species creation and side.

The channel may be defined in terms of its cross-sectional dimension or depth as well as the overall length of the channel. The cross-section and length may vary to provide an internal volume based on the application. In certain applications, the channel may be cylindrical or cubic shape. In certain applications the volume of the vessel, filter or purifying element may be between approximately 0.01 to 10000 μl. In other embodiments, the volume of the vessel may be between approximately 1 to 1000 μl.

In certain embodiments, the filtration device may be used for the purification of beta+ and beta− emitting isotopes including, but not limited to those used in nuclear medicine for diagnostics, such as PET, SPECT, and nuclear therapy. Such isotopes include ¹⁸F, ¹¹C, ¹⁴C, ^{99m}Tc, ¹²³I, ¹²⁵I, ¹³¹I, ⁶⁸Ga, ⁶⁷Ga, ¹⁵O, ¹³N, ⁸²Rb, ⁶²Cu, ³²P, ⁸⁹Sr, ¹⁵³Sm, ¹⁸⁶Re, ²⁰¹Tl, ¹¹¹In, or combinations thereof. Preferred isotopes include those used for PET such as ¹⁸F, ¹¹C and ⁶⁸Ga.

In certain embodiments, the filtration device may be used with other devices, including microfluidic devices, for the production and storage of radiopharmaceuticals containing said radioisotopes. As such the filtration device may be used in an in-line system, in fluid communication with a microfluidic reactor or storage vessel. In other embodiments, the filtration device may be used separately whereby a radioisotope is added to the device having an inlet and outlet opening.

In certain embodiments, the filtration device may be used for filtration and purification of radiopharmaceutical production, such as but not limited to radioisotope carrying tracers. Autoradiolysis in radiotracer synthesis and production is present during purification of a target compound. Quartz microfiber filters (QMA), Sep-Paks® (Waters Corporation, Milford, Mass.) solid phase extraction (SPE), liquid chromatography (LC), high pressure liquid chromatography (HPLC), or thin layer chromatography (TLC) columns and chambers may be utilized for purification and separation as well as concentrating the radiopharmaceutical compound of interest. The solid state resins used in such methods may create a high local concentration of radioactive material, leading to heavy radiolysis in said areas. By a geometric re-designing of these resins, autoradiolysis may be reduced, wherein the confining geometries, or channels, have at least one characteristic dimension below the beta+/beta− range of radioisotopes in use.

In certain embodiments, the filtration device may be a conventionally packed cartridge or column containing a solid support resin with dimensions below the beta+/beta− range of radioisotopes in use. FIG. 1 is an illustration of one embodiment, showing a cylindrical column with fluid confining geometries configured as segmented channels. FIG. 2 illustrates an embodiment wherein the confining geometries are small through-holes with characteristic inner diameters that have at least one characteristic dimensions below the beta+/beta− range of radioisotopes in use.

In still another, embodiments, the filter device may be a wrapped structure as shown in FIG. 3, wherein the channel dimensions are related to the spacing between the layers. In certain other embodiments the fluid confining geometry maybe a sponge-like or porous substrate with inner channels, chambers, conduits or fluid confinements with a characteristic dimension below the beta+/beta− range of radioisotopes in use as well as a functional surface coating allowing purification and/or concentration of radiopharmaceutical compounds or radioisotopes.

In each embodiment, the filtration device may comprise a functional surface coating or solid support for purification, phase transfer, concentration of the radioisotope or radiopharmaceutical compound, or combinations thereof. The functional surface coating and solid state resin are those generally used in separation/purification systems, including but not limited to, QMA, SEP-Paks, SPE cartridges, LC, HPLC, and TLC.

The solid support may be any suitable solid-phase support which is insoluble in any solvents to be used in the method but to which selective component of the filtrate solution may be bound. Examples of suitable solid support include polymers such as polystyrene (which may be block grafted, for example with polyethylene glycol), polyacrylamide, or polypropylene, or glass or silicon coated with such a polymer. The solid support may take the form of small discrete particles such as beads or pins, or as coatings on a particle, for example, of glass or silicon, or a coating on the inner surface of a cartridge or microfabricated device such as one or multiple microfluidic channels.

For example, [¹⁸F]-fluoride (fluorine-18) is useful for preparation of radiopharmaceuticals by nucleophilic fluorination, specifically for use in Positron Emission Tomography (PET).

Fluorine-18 is obtained by a variety of nuclear reactions from both particle accelerators and nuclear reactors, and can be produced at specific activities approaching 1.71×109 Ci/mmol. The half-life of fluorine-18 is 109.7 minutes, relatively long in comparison with other commonly used radioisotopes but still imposing time constraints on processes for preparing 18F-labelled radiopharmaceuticals.

Fluorine-18 may be produced by irradiation of an [¹⁸O] oxygen gas target by the nuclear reaction ¹⁸O(p,n)¹⁸F, and isolated as [¹⁸F]fluoride ion in aqueous solution. It also may be produced by exposing the target to H₂¹⁸O and irradiating. In aqueous form, [¹⁸F]fluoride can be relatively unreactive, and so certain steps are routinely performed to provide a reactive nucleophilic [¹⁸F]fluoride reagent. Following irradiation, a positively charged counterion is added, most commonly potassium complexed by a cryptand such as Kryptofix 222 (4,7,13,16, 21,24-hexaoxa-1,10-diazabicyclo [8,8,8] hexacosan), or alternatively, cesium, rubidium, or a tetralkylammonium salt. This is commonly achieved by passing the [¹⁸F] fluoride target water (typically in volumes of 1 to 5 mL) through an anion exchange resin and eluting with a slightly aqueous organic solution (typically in a volume of 0.1 to 5 mL) of the counterion, for example, with a potassium carbonate/Kryptofix solution in water/acetonitrile. Secondly, the solution is dried, commonly by azeotroping in the presence of a low-boiling solvent such as acetonitrile.

Automated radiosynthesis apparatus routinely include such a drying step, typically lasting 9 minutes in the case of [¹⁸F]FDG synthesis on Tracerlab MX (GE Healthcare). The compound to be labeled (dissolved in an organic solvent suitable for performing the subsequent radiosynthesis, usually an aprotic solvent such as acetonitrile, dimethylsulphoxide or dimethylformamide) is then added to the dried residue of [¹⁸F]fluoride and counterion.

By using a filtration device as described above, filtration through the device may allow rapid, trapping and elution of [¹⁸F]fluoride from target water using a solid support system. Exemplary materials are described in WO 2009/083530, incorporated herein by reference.

Purification, phase transfer and or concentrating of a radioisotope may be executed in serial manner or via parallel capillary channels. The channels comprise a proximal end and a distal end to allow fluid movement. In other embodiments, the channel may comprise a single opening wherein fluid transfer into and out of the vessel occurs through the same opening. Dimensions are dependent on the emitted beta+/beta− energy of the utilized radioisotope during decay and the resulting maximum beta+/beta− range. For example, for ¹⁸F, the maximum range for the positrons emitted in water is 2.3 mm. Therefore embodiments for the purification, reactor or storage vessel may comprise fluid confining geometric arrangements with a characteristic size below 2.3 mm for use with ¹⁸F.

In other embodiments the fluid confining geometric structure maybe a thin film or surface coating along the channel with at least one characteristic dimension below the beta+/beta− range of radioisotopes in use.

In certain embodiments, the characteristic dimensions of the fluid confining geometric structures for the filter device may be defined based on the specific beta+/beta− emitters in use. This is shown but not limited to the values displayed in Table 1, which list maximum and average range of positrons in water for several commonly used medical isotopes.

TABLE 1
Maximum and average range of positrons
in water for common medical isotopes

| | Range in | Average range |
|---|---|---|
| Radionuclide | water [cm] | in water [cm] |
| C-11 | 0.39 | 0.103 |
| C-14 | 0.028 | 0.013 |
| N-13 | 0.51 | 0.132 |
| O-15 | 0.8 | 0.201 |
| F-18 | 0.23 | 0.064 |
| P-32 | 0.785 | 0.198 |
| Rb-82 | 1.65 | 0.429 |

In certain embodiments the filtering device may have a channel width in the range of about 0.01 μm to 3000 μm and in another embodiment the channel depth may range from about 1 μm to 2000 μm. It is understood that the channel cross-section may be essentially cylindrical, oval or rectangular in shape or combinations thereof. The length of the channel is arbitrary in that it is chosen based on required volume capacity or flow.

The channels may be positioned as to provide a high packaging density. As such, geometries of the filtering device may include capillaries and capillary-like assemblies such as cylindrical or cubic shapes as well as geometries with meander-shaped, planar rectangular, coin-shaped structures or combinations thereof.

In designing for low space consumptions, positron emission and interaction to adjacent channels must be considered. For example, re-entering probabilities and energies for positrons emitted by ¹⁸-fluoride decay to adjacent channels has been calculated and estimated to show a small to negligible effect (Table 2). The results have been experimentally validated utilizing a shielded capillary setup (re-entering suppressed by appropriate shielding) and an on-chip meander structure (channel: 500 μm×500 μm, 250 μm spacing, material: COC 6017-SO4, illustrated in FIG. 4) with no measurable difference in results between the two configurations as shown graphically in FIG. 5. More specifically, as shown in FIG. 5, there is no significant difference in autoradiolysis between the two systems; hence the results suggest that there is no significant positron interaction between adjacent channels in a meander-shaped device with the present configuration.

TABLE 2
Interaction between adjacent geometric structures carrying radioactive
compounds on the example of planar meander structures and
[18F]FDG at 14.9-23.1 GBq/ml (FIG. 1)

| | | | | | Rel. | |
|---|---|---|---|---|---|---|
| Channel | Channel | Channel | Channel | Nr. of | Energy | Total |
| width | height | Spacing | Volume | parallel | increase | energy |
| [μm] | [μm] | [μm] | [μl] | channels | [%] | flux [%] |
| 250 | 500 | 250 | 200 | 56 | +1.4 | 34.5 |
| 250 | 250 | 150 | 200 | 88 | +1.5 | 29.7 |
| 500 | 500 | 250 | 200 | 32 | +1.3 | 49.0 |
| 250 | 250 | 250 | 200 | 80 | +1.0 | 29.7 |
| 250 | 250 | 500 | 200 | 66 | +0.4 | 29.1 |
| 750 | 750 | 500 | 200 | 17 | +0.5 | 62.1 |
| 500 | 500 | 500 | 200 | 28 | +0,.6 | 48.3 |
| 500 | 500 | 750 | 200 | 14 | +0.2 | 47.9 |

Even though impact of positron interaction between adjacent structures has shown no significant impact for 18-fluoride with activity concentrations between 4.3 and 23.1 GBq/ml, in certain embodiments, shielding between adjacent fluid confining geometries may be of interest for beta+/beta− radiation with higher energies than ¹⁸F or for activity concentrations higher than the evaluated amounts.

As such, in certain embodiments, the fluid confining geometry is configured such that the whole geometry or a given segment of the geometry is substantially isolated from its nearest neighbor geometry or neighbor segment such that no measurable kinetic positron energy transfer occurs between the fluid confining geometries or segments. Measurable positron energy transfer between channels refers to a shift in overall autoradiolysis suppression towards decreased values for decreasing channel spacing.

In certain embodiments a substrate material utilizing heavy materials that lead to high positron absorption and decrease the mean path length of positrons may be used. Materials for use in shielding includes usually solid or liquid materials of high density or mass or both, such as but not limited to lead, tungsten, epoxy and material combinations involving elements that lead to high beta+/beta− range damping or absorbance.

In certain embodiments shielding between adjacent fluid confining geometric structures may be achieved with absorbing material inserts between these structures (inlets). In other embodiments, design of adjacent or intermediate compensation structures such as channels or cavities filled with water or other fluids that lead to positron path length reduction or scattering may be used to reduce autoradiolysis induced between neighbor structures. The same shielding fluids may be utilized for heating and cooling of the structures that carry/transport the radioactive and non-radioactive reagents.

In certain embodiments, the purification device may be replaced by a segmented flow type arrangement for use with fluid volumes on the order of microliters to picoliters. In such embodiments, the outer dimensions of the respective droplets and the distance between these droplets define the characteristic dimensions for autoradiolysis reduction. In certain other embodiments, device is replaced by solid phase based surface chemistries. Solid phase based surface chemistries include, but is not limited to, chemistry on a frit or a functional surface, floating liquid films, interfacial chemistries and other assemblies wherein a thin layer of the radioactive compound may be included. In such embodiments the thin film shows characteristic dimensions below the beta+/beta− interaction range which leads to autoradiolysis reduction.

In certain embodiments, the filtration device may be used for the purification or concentration of radiopharmaceuticals. The method may comprise adding a mixture of a radioisotope containing compound, such as a radiotracer and a pharmaceutical carrier, to the filtration device. The mixture would be added and allowed to flow through the channels of the filtration device and collected. The filtration device would be designed such that the volume of the channel is controlled to provide adequate residence or flow through time through the filtering system. The radioisotope containing compound may be a compound containing radioisotopes such as ¹⁸F, ¹¹C, ¹⁴C, ^{99m}Tc, ¹²³I, ¹²⁵I, ¹³¹I, ⁶⁸Ga, ⁶⁷Ga, ¹⁵O, ¹³N, ⁸²Rb, ⁶²Cu, ³²P, ⁸⁹Sr, ¹⁵³Sm, ¹⁸⁶Re, ²⁰¹Tl, ¹¹¹In, or combinations thereof. Preferred isotopes include those used for PET such as ¹⁸F, ¹¹C and ⁶⁸Ga.

The pharmaceutical carrier refers to a composition which allows the application of the agent material to the site of the application, surrounding tissues, or prepared tissue section to allow the agent to have an effective residence time for specific binding to the target or to provide a convenient manner of release. The carrier may include a diluent, solvent or an agent to increase the effectiveness of the radiopharmaceutical produced. As such the carrier may also allow for pH adjustments, salt formation, formation of ionizable compounds, use of co-solvents, complexation, surfactants and micelles, emulsions and micro-emulsions. The pharmaceutical carrier may include, but is not limited to, a solubilizer including water, detergent, buffer solution, stabilizers, and preservatives.

The invention may enable synthesis to occur at an increased activity and high reagent concentration levels by appropriate design of respective channel assemblies. Issues of radiotracer synthesis at high activity levels have been reported with comparably low yield [Santiago J. et al: Reactor scale effects on F-18 Radiolabeling; 18th ISRS, Edmonton, Canada, Jul. 12-17 2009, Poster]. With an appropriate system design utilizing geometric structures as described may improve yield due to decrease in autoradiolysis. In certain embodiments the improvement may be obtained during synthesis including for example but not limited to radiolabeling, hydrolysis, purification (e.g. SEP Pack or QMA cartridge), reformulation and concentration.

In certain embodiments, the device may be used for reduction of autoradiolysis in radioisotope containing compounds productions, including for example radiotracer production and autoradiolysis which may be especially present during purification of the target compound. Usually, QMA, SEP-Paks, SPE cartridges, LC, HPLC, and TLC methods are utilized for cleaning, purification and separation. The solid state resins used in such methods create a high local concentration of radioactive material, leading to high radiolysis. By specifying the geometric design of the device, autoradiolysis can be reduced. This applies for conventionally packed cartridges and columns using geometric confining element having dimensions below the beta+/beta− range of radioisotopes in use.

In certain embodiments, the filtration device may be structures and capillaries on-chip or off-chip or inside a bulk material containing functional surface coatings or resins for purification, phase transfer and concentration of radioisotope containing material such as, but not limited to radiopharmaceuticals.

Autoradiolysis which is created by interaction of radicals may also be reduced by surface modifications to getter radicals that lead to a permanent or temporary capturing/binding of radicals to a surface. Due to short diffusion lengths for particles in micro-channels, the probability of a radical reaching the wall a capillary tube or a microfluidic structure before interacting with a radiolabeled molecule of interest is higher than compared to a conventional vessel.

In certain embodiments, the device may further comprise a device for collecting and transferring the radioisotopes. For example, the device may be designed such that in in fluid communication with another element, that can be used for transferring or storing the radioisotopes prior to its end use. In certain embodiments, the device may be part of an assembly which is loaded and unloaded utilizing high gas or fluid pressure,

### Modeling Studies

¹⁸F decays in 97% of cases to ¹⁸O via and β⁺ emission and vₑ in 3% of cases via electron capture (Cherry S, Sorenson J, Phelps M, Physics in Nuclear Medicine, Saunders (2003)). During a β⁺ decay event, a proton decays into a neutron, a positron, and a neutrino, with the difference between the binding energy and the energy converted into mass, shared between the kinetic energy of the positron and the neutrino and, less often, a photon. Neutrinos interfere only very weakly with surrounding matter, and it is reasonable to ignore their effects in the autoradiolysis process, just as it is justifiable to neglect the statistically less likely decay process of ¹⁸F electron capture. In contrast, a positron of high energy is relevant as it can directly lead to a chain of ionization events in the process of dissipating its kinetic energy.

An intact [¹⁸F]FDG molecule can lose the ¹⁸F atom if it is ionized directly by a positron or hit by a radical that causes charge transfer between the two particles. At activity concentrations of <20 GBq/ml [¹⁸F]FDG in water, the probability of a positron ionizing intact [¹⁸F]FDG molecules directly is estimated as <1% based on molar concentrations of active compounds versus water molecules. For this reason, the dominant mechanism for autoradiolysis is the interaction of radical species with intact [¹⁸F]FDG molecules. Buriova et al. have reported that the post-autoradiolytic HPLC-MS and TLC analysis showed that OH and O₂ are the two species that are most likely to cause ¹⁸F release (Buriova E. et al., Journal of Radioanalytical and Nuclear Chemistry, Vol 264 No 3 (2005) 595-602). Such reactions, if occurring with enough kinetic energy, lead to electron exchange and subsequent breaking of e.g. ¹⁸F bonds. Hence, autoradiolysis can be characterized based on the radiochemical purity (RCP) of a radiotracer solution which is determined by measurements of free ¹⁸F versus intact [¹⁸F]FDG molecules utilizing thin layer chromatography (TLC) or high pressure liquid chromatography (HPLC) coupled with a radiation detector (radio-HPLC).

The energy spectrum of the ¹⁸F decay has been studied and the kinetic energies of the positron have been determined to be Eₘₐₓ=0.633 MeV and a mean energy Eₘₑₐₙ1≈⅓Eₘₐₓ=0.211 McV. After the release of the positron, its kinetic energy is dissipated via ionization, inelastic excitation, and positronium formation which after annihilation subsequently leads to the release of two γ photons, each with an energy of E₆₅ =511 KeV. The distance in water where 90% of this γ radiation is deposited is approximately 24 cm which is much larger than the discussed geometries for the device design <2 cm. Thus, the contribution of 511 keV γ radiation to ionization can be neglected in the autoradiolysis model. Furthermore, for positrons with kinetic energies of the ¹⁸F decay spectrum the energy losses due to radiation processes are negligible (Cherry S, Sorenson J, Phelps M, Physics in Nuclear Medicine, Saunders (2003)).

The energy transferred to the ¹⁸O daughter nucleus due to momentum conservation after a positron release, including relativistic considerations, has a maximum of approximately 31 eV since the mass ratio of a positron to an ¹⁸O atom is ˜10⁵. Lapp and Andrews reported the mean ionization energy for water as 68 eV and the lowest ionization energy as 11.8 eV (Lapp, Andrews, Nuclear Radiation Physics, Prentice Hall, 1972, p. 154). This means that the recoil effect of positron emission on the daughter nucleus with max. 31 eV has negligible effect on autoradiolysis when compared to the direct effect of the positron which has an average energy in the range of 230000 eV.

It is assumed that the fraction H(r) of the total energy lost by the positron each time it collides and ionizes is approximately constant for all distances r from the daughter nucleus. Furthermore, it is assumed that the number of ions produced is proportional to the energy lost as ionization energy, and that the number of ¹⁸F atoms released correlates linearly on the number of positron-generated radicals in solution. Ionization energy is hereby defined as the energy that is lost by a positron during ionization of an atom. In general, not all the positron energy is lost to overcome the binding energy of an electron but it may also be lost in secondary processes such as photon emission or as kinetic energy transferred to the emitted electron.

The model developed for the estimation of autoradiolysis effects in small geometries is based upon energy conservation considerations and represents the worst case scenario. This means that due to the assumptions made in (2.) the measured autoradiolysis should not exceed the values predicted by the model. All calculations refer to ¹⁸F decay and the corresponding positron energy levels.

When the number of ions Nᵢₒₙₛ produced is proportional to the deposited ionization energy, then Nᵢₒₙₛ can be calculated as:

Nᵢₒₙₛ(r)∝H(r), (1)

where H(r) is the fraction of energy lost due to ionization for a constant distance r and E_{absorb}(r) is the total energy deposited up to distance r. The results of Palmer and Brownell have been used for the estimation of the fraction of total deposited energy in the system (Palmer and Brownell, 1992 IEEE Trans. Med. Imaging 11, 373-8). Palmer et al. have reported that the 3D distributions of the positron annihilation events can be interpolated by the Gaussian function

$$P ( r ) = \frac{1}{σ \sqrt{2} Φ ( \frac{{r}_{0}}{σ \sqrt{2}} ) + σ \sqrt{\frac{π}{2}}} exp ( - \frac{{( r - {r}_{0} )}^{2}}{2 {σ}^{2}} ) . \qquad ( 2 )$$

Parameters r₀ and obtained by Gaussian fittings, have been reported for different isotopes. In order for P(r) to be the probability density, the normalization function Φ is introduced and defined as:

Φ(u)=∫₀^{u}e^{-r}^{²}dx, (3)

It has been shown by Champion et al. that for ¹⁸F decay r₀=0.04 mm: and σ=0.780 mm for water as the decay event surrounding medium (Champion C, Le Loirec C, Phys. Med. Biol. 52 (2007), 6605-6625). Using these fit parameters the cumulative positron annihilation probability curve, defined as

T(x)=∫₀^{x}P(r)dr, (4)

is shown in FIG. 6. This curve yields the probability that a positron from the ¹⁸F spectrum annihilates up to a certain distance x.

FIG. 6 suggests that approximately 80% of positrons annihilate after passing through a 1 mm thick layer of water. This result corresponds well with Monte Carlo simulation values reported by Champion et al. (76%) and Alessio et al. (79%) (Champion C, Le Loirec C, Phys. Med. Biol. 52 (2007), 6605-6625 and Alessio A., MacDonald L., Nuclear Symposium Conference Record, 2008)).

The range-energy relations for positrons and electrons have been broadly studied and the results from Katz and Penfold demonstrate that there is an empirical relation between the energy and the range (Katz L, Penfold A. S, Rev. Mod. Phys. 24, 28 (1952)).

For the transmission of a mono-energetic β particle beam in aluminum with an energy E₀, where 0.01 MeV≦E₀≦2.5 MeV, the following empirical relation has been postulated:

R(E′)=412·E′^{1.205-0.0954 lnt(E′)}, (5)

where the range R(E′) is expressed in (mg/cm²) whereas E′ is dimensionless, given by E′=E/MeV. Using this relationship, the range in a specific matter can be calculated by dividing the range R(E′) by the density of the matter:

$$Range ( {E}^{′} ) = \frac{R ( {E}^{′} )}{ρ} . \qquad ( 6 )$$

The empirical energy-range relation (5) can transform the cumulative annihilation probability distribution T(x) in (4), into a function that shows the fraction of total energy deposited E_{absorb}(r) up to the distance r from the daughter nucleus. In a more general form:

E_{absorb}(r)=T(r)·Range⁻¹(r), (7)

where T(r)=∫₀^{r}P(u)du is the annihilation probability and Range⁻¹ denotes the inverse function Range(E).

A rigorous derivation of equation (7) should consider backscattering, however, the work of Kobetich and Katz justify that backscattering can be neglected in this case (Kobetich R., Katz L., Physical Review, Vol 170 No 2, 1968).

The normalized dissipation energy curve for positrons in water based on (7) is shown in FIG. 7. Water is chosen as the medium since injectable radiopharmaceuticals are usually aqueous solutions.

It can be seen from the FIG. 7 that about 85% of the positrons kinetic energy is deposited in the first 1 mm of the surrounding water and only 13% within the first 100 μm. Following the assumption that the autoradiolysis phenomena is linearly proportional to the number of ions in solution, and that the number of ions created is proportional to the amount of energy deposited in the system as ionization energy E_{absorb}(r) (see 2.), the results suggest that autoradiolysis effects can be reduced to approx. 30% by tailoring the geometry to λₚₐₜₕ=250 μm. This means a reduction by 70% in comparison to conventional geometries where the mean path length is approximately equal to the positron's range λₚₐₜₕ≈R with R=2.3 mm for ¹⁸F.

### Application to Cylindrical and Planar Systems

A general cylindrical system suitable for analysis with the previously developed model is described by a cylinder with length L and radius r, such that L>>r. This approximation allows end-effects to be neglected. A further constraint for model applicability is that the cylinder is shielded or otherwise configured in a way such that a positron leaving the cylinder cannot reenter at another location.

The mean path length may be defined as the average distance of a positron traveling inside a given configuration of geometric boundaries such as a cylinder or a planar structure, taking multiple starting positions and directions in a three dimensional geometry into account. The mean path length correlates with the energy dissipated inside a geometric configuration. Hence, the mean path length represents the link between the autoradiolysis model of positron energy dissipation (FIG. 4) and the actual geometric configuration explored.

To calculate the mean path length as a function of the cylinder's radius for positrons emitted during ¹⁸F decay and their respective energy distribution and range, a Monte Carlo simulation was executed with 100,000 positrons for each cylinder radius varying between 0 to 2.3 mm. The result of the simulation is displayed in FIG. 8

The mean path length for device consisting of two wide thin sheets (FIG. 9, a being the length, b the width and c the distance between the bottom and top layer of the rectangular chamber, such that a>>c, b>>c) was also examined utilizing a Monte Carlo simulation. For each distance between the sheets, the simulation has been run with 100,000 positrons and the results are displayed in FIG. 8. Circular embodiments instead of the present rectangular example are expected to show similar results for energy deposition and resulting autoradiolysis.

With the mean path lengths for the cylindrical (FIG. 8) and planar (FIG. 10) configurations determined, the fraction of kinetic positron energy deposited into a fluid inside these geometric configurations can be calculated according to (7). Characteristic dimensions are the radius r for the cylinder and the thickness c for the planar geometry. The results are displayed in FIG. 8. The maximum characteristic dimension where E_{absorb}=100% was set to r=c=2.7 mm for both configurations.

The results show that both geometric arrangements can be used for autoradiolysis reduction, if the characteristic dimensions are chosen small enough. With the assumption of Nᵢₒₙₛ∝E_{absorb} the results in FIG. 11 suggest that a cylindrical capillary with radius r=250 μm results in a comparative level of autoradiolysis not exceeding 36% of the bulk device configuration with r=2.7 mm. Furthermore it can be concluded that cylindrical-like systems offer a higher potential for autoradiolysis reduction than planar shapes. In contrast, planar structures offer an increased packaging density and lower absolute internal surface area, both being potentially important parameters during system design.

The model assumes that a positron loses a constant fraction of its instantaneous kinetic energy due to ionization, independent of the distance to the decaying atom. Upon first inspection, this approximation seems to be bold, since the total ionization cross-section for positrons in water is a complex function of the kinetic energy. The claim can be justified by considering not only the ionization cross-section but also cross-sections associated with the dissipative processes of inelastic excitations and positronium formation. Using the results of Champion et al. it can be shown that for positron energies >1 keV, the ionization fractional cross-section is almost constant at ˜80% (Champion C, Le Loirec C, Phys. Med. Biol. 52 (2007), 6605-6625).

### Experimental

### Materials & Methods:

The autoradiolysis trends predicted by the theoretical model were evaluated experimentally by synthesizing non-stabilized [¹⁸F]FDG and distributing the product into a variety of geometries. A GE TRACERlab MX synthesizer (GE Healthcare, Liege, Belgium) together with TRACERlab MX_{FDG} cassettes (Cat.No: PS150ME, GE), the [¹⁸F]FDG reagents kit (Prod. No.: K-105™, ABX, Radeberg, Germany) and Mannose Triflate plus (Prod. No.: 107.0025, ABX) were utilized for synthesis. A GE PETtrace cyclotron (GE Healthcare, Uppsala, Sweden) was used to irradiate two silver targets with 1.6 ml of H₂¹⁸O each (dual beam mode) for up to 90 minutes at 35 μA for each target to generate ¹⁸F-activity of up to ca. 200 GBq. The standard [¹⁸F]FDG synthesis protocol and cassette was modified to avoid introduction of ethanol into the process (ethanol vial in cassette replaced by empty flask). Prior to synthesis, two C18-cartrigdes were removed from the cassette and manually conditioned with 10 ml of ethanol, 20 ml of water, dried with air and subsequently reassembled into the cassette. A total number of ten syntheses were performed, each producing 4 ml of [¹⁸F]FDG at activity concentrations between 4 GBq/ml and 23 GBq/ml. No ascorbic acid, ethanol nor other stabilizers were added prior, during or after synthesis. The synthesis output was examined for residual ethanol by GC-MS (6890N Network GC-System with MS 5975B, Agilent Technologies, Germany).

The synthesis product was then distributed using an automated experimental set-up as shown in FIG. 12. The autoradiolysis reduction effect of a thin cylindrical geometry was explored using 1/16″ outer diameter PEEK capillaries with inner diameters from 250 μm inner diameter (ID) to ID 750 μm, whereas 200 μl of product was injected into each capillary. The capillary length was varied to keep a constant internal volume of 200 μl. The capillaries were wrapped around a steel core of 15 mm diameter, in a spiral with a pitch of 4 mm The spiral wrapped capillaries were shielded by 3 mm of aluminum. The shielded spiral configuration ensured that positrons leaving the capillary had no opportunity to re-enter a segment of the adjacent capillary.

Autoradiolysis suppression was defined as the reduction in autoradiolysis relative to a 300 μl sample stored in a bulk reactor. The bulk reactor result was created from storage of non-stabilized [¹⁸F]FDG in a 2 ml glass vial which was part of the capillary filling routine. The results observed in a bulk reactor may be correlated to residence time within a microfluidic filtration device compared to a bulk filtration device.

The capillary filling routine also included a first step and a last step where 300 μl of [¹⁸F]FDG was dispensed into a vial with 15% ethanol solution present. These two samples were taken in order to evaluate the impact of the capillary filling time (about 20 min to 30 min) on the final autoradiolysis result after 14 hours, since the autoradiolysis rate is at its maximum directly after synthesis [16].

After 14 hours, the capillary contents were ejected into separate vials utilizing H₂O and subsequently the ratio of free ¹⁸F to [¹⁸F]FDG in for each capillary output solution and all bulk vial standards was determined. TLC (Polygram SIL G/UV 254; Macherey-Nagel) and an autoradiograph (Phosphor-Imager Cyclone Plus, PerkinElmer, Germany) were used to quantify the ratio of free ¹⁸F to [¹⁸F]FDG which also known as radiochemical purity (RCP).

### Results:

The autoradiolysis suppression for all experiments is summarized in FIG. 13. It was calculated for all runs from the respective RCP of the 300 μl glass vial reference sample (worst case, 0% autoradiolysis suppression after 14 hours) to the initial RCP after synthesis (best case, minimum autoradiolysis). FIG. 13 shows that an ID 250 μm capillary provides an autoradiolysis suppression of >90% whereas an increasing capillary diameter results in a reduction of the suppression factor which is in general agreement with the trend predicted by the model.

The ethanol content was measured to <2 mg/l ethanol for all experiments (detection limit of the instrument). The difference in autoradiolysis between the 300 μl ethanol stabilized samples taken prior and after capillary filling was measured <1%, suggesting that the filling time had no impact on the final results.

FIG. 14 displays experimental results for the autoradiolysis suppression inside an ID 250 μm capillary (n=9) versus the respective activity concentration for each run. There are no significant trends suggesting that the results displayed in FIG. 14 are comparable for the activity concentrations chosen.

Apart from activity concentration, the results of FIG. 14 may have been affected by permanent immobilization of free ¹⁸F on the inner capillary surface. In order to investigate this aspect for the present configuration of tubing and materials, the capillaries were flushed with 400 μl of water after each experimental run and the rinses were analyzed by TLC. Water has shown to be very effective for cleaning residual activities from capillary tubing. The results yielded similar ratios of ¹⁸F to [¹⁸F]FDG as the original capillary contents (variation of +/−3%) and provided no evidence for the capillary acting as a ¹⁸F trap. However, temporary surface immobilization effects for ¹⁸F as well as permanent or temporary immobilization of free radicals may have an effect and cause the discrepancy between the model (linear correlation with capillary diameter) and experimental results (non-linear correlation with capillary diameter). According to the theoretical results of the cylinder, planar devices with appropriate dimensions would show comparable results.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A device comprising:
two or more confining geometries, said confining geometries comprising;
a cross-section dimension below the beta(+) or beta(−) range of a radioisotope, when containing the radioisotope; and
wherein adjacent confining geometries are configured such that neighboring confiding geometries are isolated from the nearest neighbor confining geometry such that no measurable kinetic positron energy transfer occurs between the confining geometries when containing the radioisotope;
an inlet to allow fluid transfer in to said confining geometries; and
an outlet for to allow fluid transfer out of said confining geometries.

2. The device of claim 1 wherein the beta(+) or beta(−) range is about 0.01 μm to 3000 μm

3. The device of claim 1 wherein the beta(+) or beta(−) range is about 1 μm to 2000 μm.

4. The device of claim 1 wherein the confining geometries comprises a rectangular, triangular, or circular cross-section channel or combinations thereof.

5. The device of claim 1 wherein the confining geometries comprises the spacing between a wrapped layer structure.

6. The device of claim 1 wherein at least one of the confining geometries or the area between the confining geometries are comprised of a high positron absorption material.

7. The device of claim 6 wherein the high positron absorption material is lead, tungsten, epoxy, or a combination thereof.

8. The device of claim 1 further comprising a solid support positioned within the confining geometry.

9. The device of claim 8 wherein the solid support comprises a polymer, glass, silicone, or combination thereof capable of binding one or more components of the radioisotope containing mixture.

10. The device of claim 1 wherein the confining geometries further comprises a functional surface coating for purification, phase transfer and concentration of a radioisotope containing material.

11. The device of claim 1 further comprising a shielding structure positioned between adjacent confining geometries.

12. The device of claim 11 wherein the shielding structure comprises a positron absorption material insert, and a positron absorption fluid, or a combination thereof.

13. The device of claim 1 wherein the device is a quartz microfiber filter (QMA), solid phase extraction cartridges (SPE), liquid chromatography column (LC), high pressure liquid chromatography column (HPLC), thin layer chromatography chamber (TLC) or a combination thereof.

14. The device of claim 1 wherein the device is further configured for loading and unloading radioisotopes for end use applications.

15. The device of claim 1 wherein the radioisotope comprises ¹⁸F, ¹¹C, ¹⁴C, ^{99m}Tc, ¹²³I, ¹²⁵I, ¹³¹I, ⁶⁸Ga, ⁶⁷Ga, ¹⁵O, ¹³N, ⁸²Rb, ⁶²Cu, ³²P, ⁸⁹Sr, ¹⁵³Sm, ¹⁸⁶Re, ²⁰¹Tl, ¹¹¹In, or a combination thereof.

16. The device of claim 15 wherein the radioisotope comprises ¹⁸F, ¹¹C, ⁶⁸Ga or combinations thereof.

17. A method comprising:
adding a radioisotope containing mixture to a device, said device comprising;
two or more confining geometries, said confining geometries comprising;
a cross-section dimension below the beta(+) or beta(−) range of a radioisotope, when containing the radioisotope; and
wherein adjacent confining geometries are configured such that neighboring confiding geometries are isolated from the nearest neighbor confining geometry such that no measurable kinetic positron energy transfer occurs between the confining geometries when containing the radioisotope;
an inlet to allow fluid transfer in to said confining geometries;
an outlet for to allow fluid transfer out of said confining geometries; and
a solid support or surface coating positioned within the confining geometry;
flowing the mixture through the device wherein the flow rate is controlled to separate, purify, or concentrate the radioisotope compound from the mixture ; and
collecting an eluent from the outlet port of the device wherein said eluent comprises the radioisotope.

18. The method of claim 17 wherein the radioisotope comprises ¹⁸F, ¹¹C, ¹⁴C, ^{99m}Tc, ¹²³I, ¹²⁵I, ¹³¹I, ⁶⁸Ga, ⁶⁷Ga, ¹⁵O, ¹³N, ⁸²Rb, ⁶²Cu, ³²P, ⁸⁹Sr, ¹⁵³Sm, ¹⁸⁶Re, ²⁰¹Tl, ¹¹¹In, or a combination thereof.

19. The method of claim 17 wherein the device is a quartz microfiber filter (QMA), solid phase extraction cartridges (SPE), liquid chromatography column, (LC), high pressure liquid chromatography column (HPLC), thin layer chamber (TLC) or a combination thereof.

20. The method of claim 17 wherein the solid support comprises a polymer, glass, silicone, or combination thereof capable of binding one or more components of the radioisotope containing mixture.

21. The method of claim 16 wherein the device further comprising a shielding structure positioned between adjacent confining geometries.
